(19) United States
(12) Patent Application Publication
(10) Pub. No.: US 2023/0395265 A1
(43) Pub. Date: Dec. 7, 2023

(54) BLOOD INFORMATION ESTIMATING APPARATUS

(71) Applicant: NTT DOCOMO, INC., Chiyoda-ku (JP)

(72) Inventors: Yuuki IKEZOE, Chiyoda-ku (JP); Takafumi YAMAUCHI, Chiyoda-ku (JP)

(73) Assignee: NTT DOCOMO, INC., Chiyoda-ku (JP)

(21) Appl. No.: 18/249,320

(22) PCT Filed: Oct. 15, 2021
(86) PCT No.: PCT/JP2021/038247

§ 371 Date: Apr. 17, 2023

(30) Foreign Application Priority Data
Nov. 19, 2020 (JP) 2020-192432

(51) Int. Cl. G16H 50/30; G16H 10/60

(52) U.S. Cl. CPC G16H 50/30; G16H 10/60

(57) ABSTRACT

A blood information estimating apparatus that determines a blood status such as a blood pressure of a user is provided. The blood information estimating apparatus 100 includes a data acquiring function 41 serving as a log acquiring unit that acquires a terminal use log of a user terminal such as a mobile terminal and a hypertension detecting function serving as an estimation unit that estimates blood information on change of a blood status based on the terminal use log. The blood information estimating apparatus 100 includes a hypertension detection model that outputs blood information based on the terminal use log. The hypertension detecting function 45 estimates the blood information using the hypertension detection model. With this configuration, it is possible to estimate a blood status of a user without imposing a burden on the user. Accordingly, it is possible to simply estimate a status such as masked hypertension.

### TECHNICAL FIELD

The present invention relates to a blood information estimating apparatus that estimates a status of a user's blood.

### BACKGROUND ART

Patent Literature 1 discloses a method of detecting change of a patient's health status. Particularly, it is described that a risk of change of a medical symptom of a patient is predicted based on a use log of a native communication application and a health risk model.

### CITATION LIST

### Patent Literature

[Patent Literature 1] Japanese Unexamined Patent Publication No. 2015-529359

### SUMMARY OF INVENTION

### Technical Problem

Among health risks, hypertension may not have subjective symptoms. Generally, when measuring blood pressure in an examination room, only one time point in the examination room is focused on. Since a blood pressure changes depending on the environment in which a subject is placed or the measurement time, health risks such as hypertension may be missed. That is, when blood pressure is continuously measured at home, it is possible to measure blood pressure which can be determined to be that of hypertension, but when blood pressure is continuously measured in an examination room or the like, it may not be possible to measure blood pressure which can be determined to be that of hypertension. This blood pressure status is called masked hypertension. On the other hand, in the technique described in Patent Literature 1, it is described that prediction of a health risk is performed, but a status such as a blood pressure is not ascertained and it is difficult to determine masked hypertension or the like.

Therefore, in order to solve the aforementioned problem, an objective of the present invention is to provide a blood information estimating apparatus that determines a blood status such as a blood pressure of a user.

### Solution to Problem

A blood information estimating apparatus according to the present invention includes: a log acquiring unit configured to acquire a use log of a user terminal; and a blood information estimating unit configured to estimate blood information on change of a blood status based on the use log.

According to the present invention, it is possible to estimate blood information such as a blood pressure without requiring active examination of a user.

### Advantageous Effects of Invention

According to the present invention, it is possible to estimate blood information without requiring active examination of a user.

### BRIEF DESCRIPTION OF DRAWINGS

FIG. 1 is a diagram illustrating a functional configuration of a blood information estimating apparatus 100 according to the present disclosure.

FIG. 2 is a diagram illustrating a functional configuration of a terminal use log acquiring unit 20.

FIG. 3 is a diagram illustrating a functional configuration of a weather information acquiring unit 3.

FIG. 4 is a diagram illustrating a functional configuration of a hypertension detecting unit 40.

FIG. 5 is a diagram illustrating a relationship between terminal use logs and life habits.

FIG. 6 is a diagram illustrating training data stored in a training data input database 40a.

FIG. 7 is a flowchart illustrating operations of the hypertension detecting unit 40.

FIG. 8 is a flowchart illustrating a process of constructing a hypertension detection model 44a.

FIG. 9 is a flowchart illustrating a hypertension detecting process using the hypertension detection model 44a.

FIG. 10 is a diagram illustrating an example of a hardware configuration of a blood information estimating apparatus 100 according to an embodiment of the present disclosure.

### DESCRIPTION OF EMBODIMENTS

An embodiment of the present disclosure will be described with reference to the accompanying drawings. The same components are denoted, if possible, by the same reference numerals and signs, and thus description thereof will not be repeated.

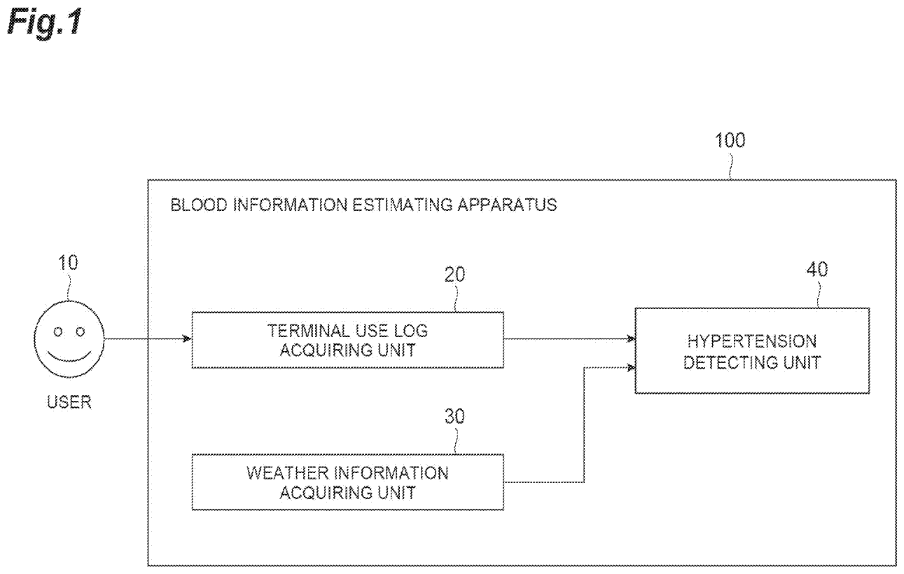

FIG. 1 is a diagram illustrating a functional configuration of a blood information estimating apparatus 100 according to the present disclosure. The blood information estimating apparatus 100 includes a terminal use log acquiring unit 20, a weather information acquiring unit and a hypertension detecting unit 40.

The terminal use log acquiring unit 20 is a part configured to acquire a terminal use log of a user terminal which is operated by a user 10 and user attribute information of the user of the user terminal.

The weather information acquiring unit 30 is a part configured to acquire weather information of an area in which the user 10 is located.

The hypertension detecting unit 40 is a part configured to detect a blood status such as a blood pressure of the user 10 based on the terminal use log, the user attribute information, and the weather information which are acquired by the terminal use log acquiring unit 20 and the weather information acquiring unit 30. In the present disclosure, hypertension is assumed to be a blood pressure status, but the present disclosure is not limited thereto. Another blood status such as a blood pressure, a blood-sugar level, a triglycerides level, and a cholesterol level may be detected in addition to the blood pressure status.

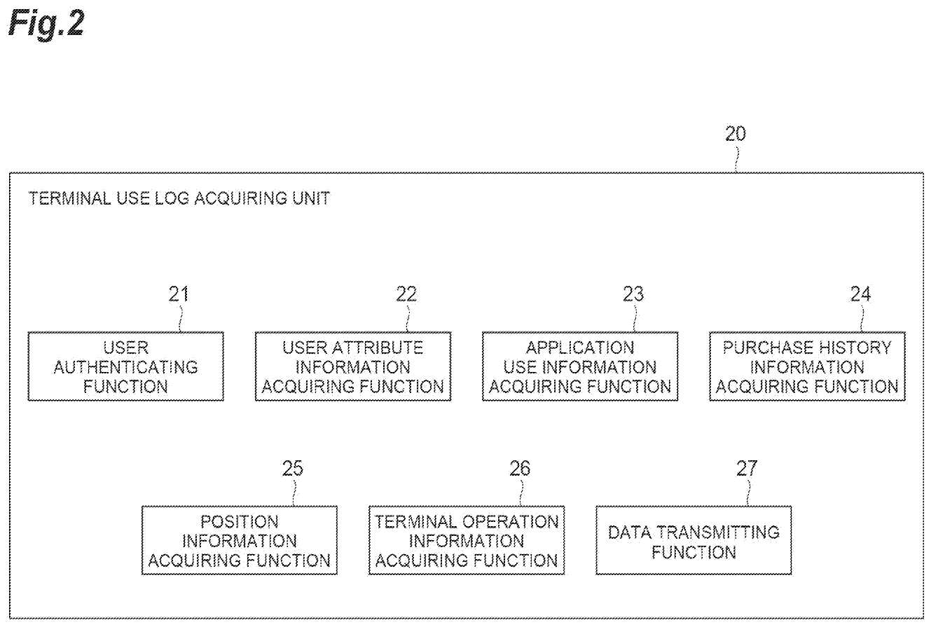

The configuration of the terminal use log acquiring unit 20 will be described below. FIG. 2 is a diagram illustrating a functional configuration of the terminal use log acquiring unit 20. The terminal use log acquiring unit 20 includes a user authenticating function 21, a user attribute information acquiring function 22, an application use information acquiring function 23, a purchase history information acquiring function 24, a position information acquiring function 25, a terminal operation information acquiring function 26, and a data transmitting function 27.

The user authenticating function 21 is a function of authenticating whether a user of a user terminal is an authorized user when user attribute information or a terminal use log is acquired from the user terminal. The user terminal is a wearable terminal (such as a wristwatch type or a spectacles type).

The user attribute information acquiring function 22 is a function of directly acquiring user attribute information from a user terminal or the user 10. The user attribute information is attribute information of a user which can affect a blood status such as age and sex of the user.

The application use information acquiring function 23 is a function of acquiring the number of steps, a sleeping time, a weight, and the like of a user acquired by an application related to healthcare (hereinafter abbreviated to a healthcare-related application) as a terminal use log. The healthcare-related application can measure the number of steps using a gyro function provided in the user terminal and/or a reported value input to the application of the user. The healthcare-related application measures behavior of the user terminal using an operation history of the user terminal or the gyro function and calculates a period in which behavior cannot be measured as a sleeping time. Alternatively, the healthcare-related application may acquire the sleeping time based on a reported value which the user has input to the application. The weight may be estimated from an image of face or the like of the user or may be acquired based on a reported value which the user has input to the application or a measured value from a measuring device cooperating with the user terminal. The application use information acquiring function 23 can measure a temperature and a pulse rate using the healthcare-related application or acquire the temperature and the pulse rate through an input from the user. The application use information acquiring function 23 can acquire a camera image captured by a camera provided in the user terminal as a use log.

The purchase history information acquiring function 24 acquires purchase history information indicating a purchase which is performed using a payment function of the user terminal as the terminal use log. The payment function is payment using a QR code or payment using a non-contact IC card. The purchase history information includes purchase store information, a purchase price, a purchased commodity, and a date.

The position information acquiring function 25 is a function of acquiring a current position of the user as the terminal use log and acquires GPS information or base station service information measured by the user terminal and a position based on a Wi-Fi access point.

The terminal operation information acquiring function 26 is a function of acquiring terminal operation information as the terminal use log. The terminal operation information is information indicating screen on/off, acceleration, illuminance, a browsing URL, a used application, and the like. Terminal operation information is stored as a history in the user terminal, and the terminal operation information acquiring function 26 acquires the history.

The data transmitting function 27 is a function of transmitting the user attribute information and the terminal use log acquired by the aforementioned acquisition functions to the hypertension detecting unit 40.

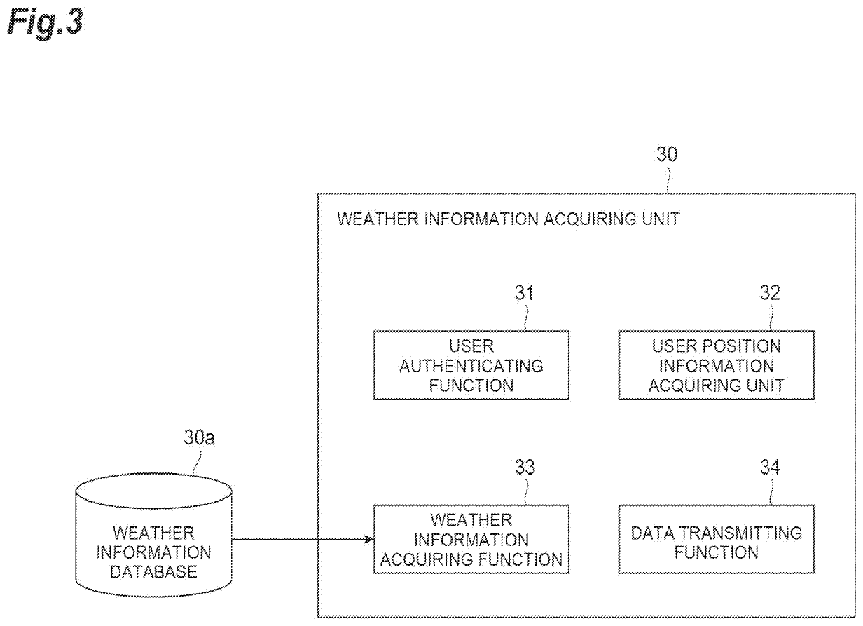

The functional configuration of the weather information acquiring unit 30 will be described below. FIG. 3 is a diagram illustrating the functional configuration of the weather information acquiring unit 30. The weather information acquiring unit 30 includes a user authenticating function 31, a user position information acquiring function 32, a weather information acquiring function 33, and a data transmitting function 34.

The user authenticating function 31 is a function of authenticating a user. When weather information is acquired, position information of the user needs to be ascertained, and authentication of the user is performed at that time.

The user position information acquiring function 32 is a function of acquiring position information of a user. The position information is acquired from a server of a mobile communication network that manages positions of user terminals or is acquired from the user terminals.

The weather information acquiring function 33 is a function of acquiring weather information based on the position information of the user from a weather information database 30a.

The data transmitting function 34 is a function of transmitting the acquired weather information to the hypertension detecting unit 40.

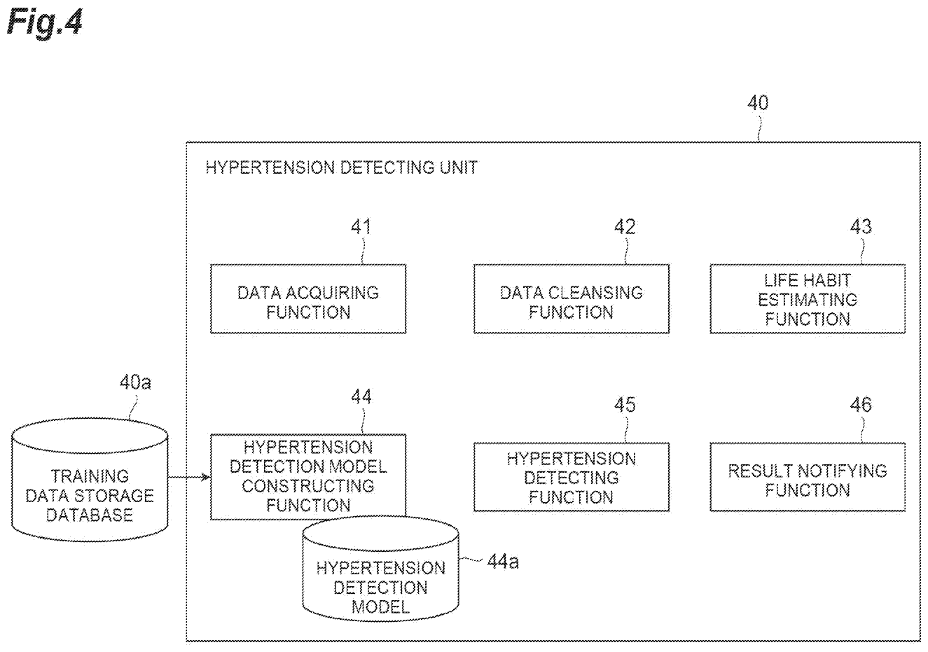

The hypertension detecting unit 40 will be described below. FIG. 4 is a diagram illustrating the functional configuration of the hypertension detecting unit 40. The hypertension detecting unit 40 includes a data acquiring function 41 (a log information acquiring unit), a data cleansing function 42, a life habit estimating function 43, a hypertension detection model constructing function 44 (a learning unit), a hypertension detecting function 45 (a blood information estimating unit), and a result notifying function 46.

The data acquiring function 41 is a function of acquiring the terminal use log, the user attribute information, and the weather information from the terminal use log acquiring unit 20 and the weather information acquiring unit 30. The data acquiring function 41 acquires the terminal use log or the like acquired in a predetermined period by the terminal use log acquiring unit 20 and the weather information acquiring unit 30 at an arbitrary timing or periodically. The user attribute information may not be collected as long as it is stored in advance. Alternatively, the user attribute information may be collected once and stored.

The data cleansing function 42 is a function of performing a process of cleansing a missing value, an abnormal value, and the like of the terminal use log and the weather information acquired in the predetermined period.

The life habit estimating function 43 is a function of estimating a life habit such as an amount of motion, commuting means/time/pattern, stress, a degree of fatigue, regularity of life/sleep, an eating-out frequency, a salt intake, and a calorie intake based on the terminal use log, the use attribute information, and the like. The life habit estimating function 43 may estimate a degree of happiness based on the terminal use log and the user attribute information. The degree of happiness is calculated by comprehensively considering the amount of motion, the commuting means and the like, the stress, the degree of fatigue, and the like.

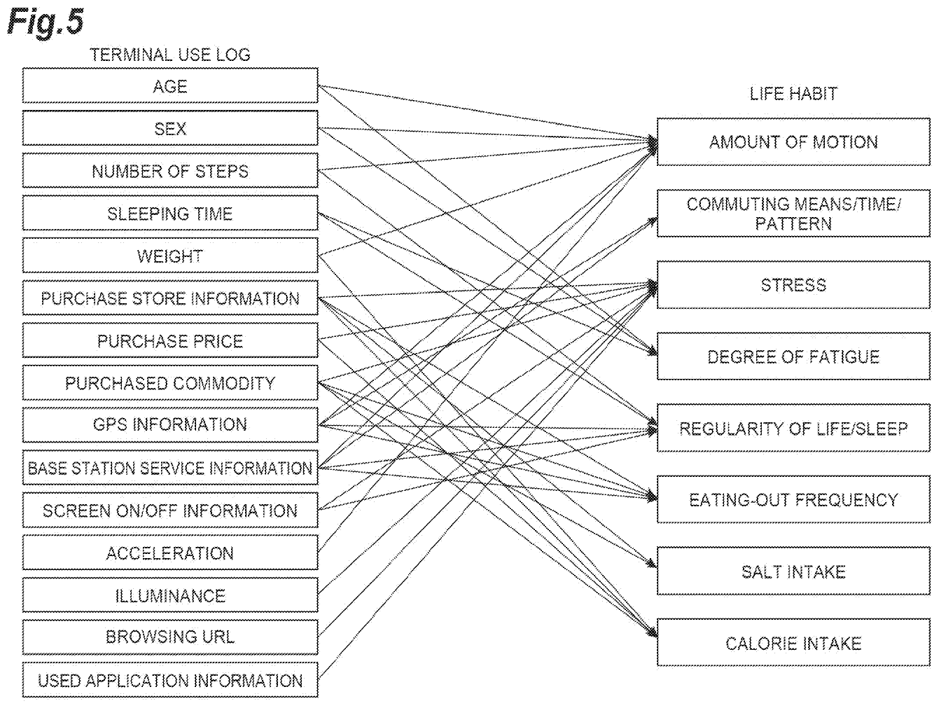

A relationship used to derive a life habit from the user attribute information and the terminal use log will be described below FIG. 5 is a diagram illustrating a relationship between terminal use logs and life habits. The life habit estimating function 43 estimates a life habit based on the relationship illustrated in FIG. 5. The amount of motion as a life habit is expressed, for example, by calorie consumption and is estimated based on age, sex, the number of steps, GPS information, base station service information, and acceleration in the user attributes and the terminal use log. The commuting means/time/pattern indicates a commuting time period or the like and is estimated based on the GPS information and the base station service information. For example, a distance between home and an office/school, a movement time, and a movement route are ascertained based on a movement time, a movement speed, a stay position, and the like, and the commuting means or the like is estimated therefrom. The stress indicates a degree of stress and is expressed, for example, in 10 levels. The stress is estimated based on purchase store information, a purchase price, a purchased commodity, screen on/off information, illuminance, a browsing URL, and used application information using a stress estimation algorithm. The fatigue indicates a degree of fatigue and is estimated based on age, sex, and a sleeping time. The degree of fatigue is estimated to be high when the sleeping time is short. The regularity of life/sleep indicates a degree of regularity and is estimated based on the number of steps, the sleeping time, the GPS information, the base station service information, and the screen on/off information. The eating-out frequency indicates the number of times of eating-out and is estimated based on the purchase store information, the purchased commodity, the GPS information, and the base station service information. The salt intake indicates a specific value or a degree of intake salt and is estimated based on the purchase store information and the purchased commodity. The calorie intake indicates a specific value or a degree of intake calorie and is estimated based on the weight, the purchase store information, the purchase price, and the purchased commodity. The aforementioned description is an example and can be appropriately modified.

The life habit estimating function 43 is a function of estimating a life habit corresponding to each terminal use log based on the terminal use logs. The life habit estimating function 43 determines a life habit using a predetermined algorithm. For example, the life habit estimating function 43 calculates an amount of motion based on the number of steps.

The hypertension detection model constructing function 44 is a function of constructing a hypertension detection model (a hypertension prediction model) based on training data stored in a training data input database 40a. The training data is collected by a healthcare application which is installed in advance in the corresponding user terminal.

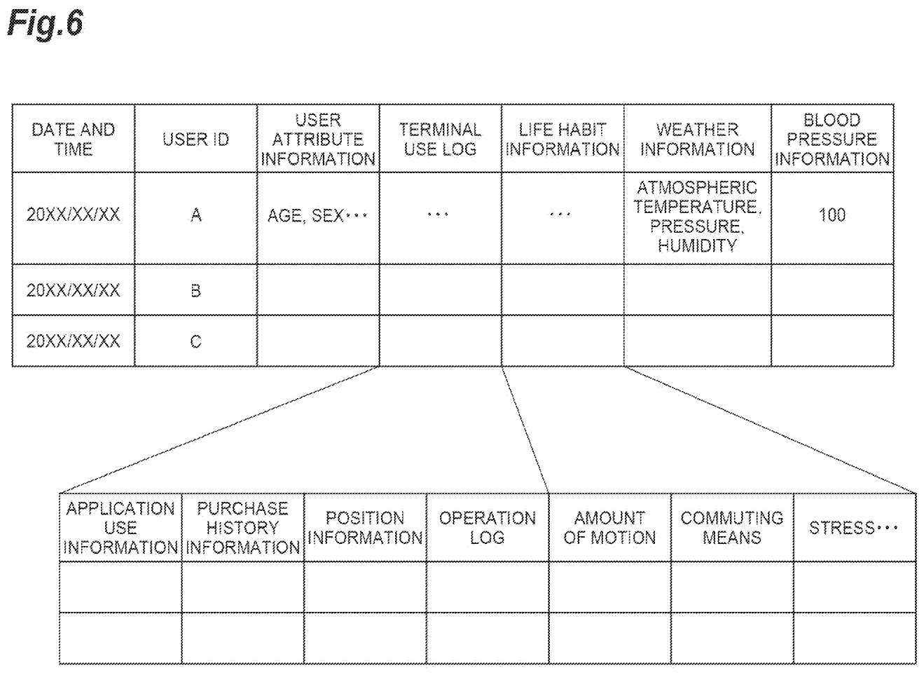

FIG. 6 is a diagram illustrating training data stored in the training data input database 40a. As illustrated in the drawing, user attribute information, a terminal use log, life habit information, weather information, and blood pressure information are stored for each user and for each date and time. The training data is data which is provided in advance from the users, and information corresponding to a predetermined period (for example, several months) is provided from the users. The hypertension detection model constructing function 44 constructs the hypertension detection model by performing machine learning using the user attribute information, the terminal use log, the life habit information, and the weather information as explanatory variables and using the blood pressure information (an average value in the predetermined period or a binary value indicating whether there is hypertension determined from the average value) as an objective variable. Blood-sugar level information, triglycerides information, or cholesterol information may be stored in addition to the blood pressure information or instead of the blood pressure information, and an estimation model may be constructed using the stored information as an objective variable.

The hypertension detection model constructing function 44 may construct a hypertension detection model 44a for each piece of user attribute information. For example, the hypertension detection model constructing function 44 may train the hypertension detection model 44a by classifying the terminal use log and the blood information for each age or/and sex.

The hypertension detecting function 45 is a function of inputting the terminal use log, the weather information, and the user attribute information acquired by the data acquiring function 41 and the life habit information estimated by the life habit estimating function 43 to the hypertension detection model, estimating blood pressure information as an output thereof, and detecting hypertension. It is important to input at least the terminal use log, and accuracy is further improved by considering the lift habit information, the weather information, and the user attribute information in addition to the terminal use log.

The result notifying function 46 is a function of notifying the user of the blood pressure information. For example, the result notifying function 46 notifies a user terminal owned by the user of the blood pressure information. The result notifying function 46 notifies of blood-sugar level information, triglycerides information, and cholesterol information when the blood-sugar level information, triglycerides information, and cholesterol information are estimated in addition to the blood information. The result notifying function 46 may notify of a life habit that can enhance a risk of hypertension of the user out of the life habits estimated by the life habit estimating function 43.

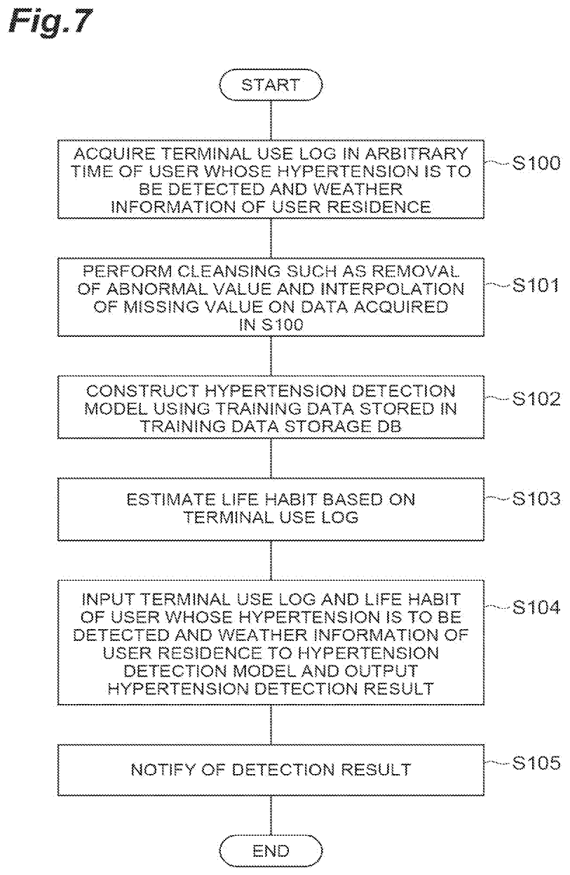

The operation of the hypertension detecting unit 40 according to the present disclosure will be described below. FIG. 7 is a flowchart illustrating the operation. The data acquiring function 41 acquires a terminal use log in a predetermined period of a user whose hypertension is to be estimated and which is acquired by the terminal use log acquiring unit 20 and weather information of a location of the user acquired by the weather information acquiring unit 30 from a user terminal (S100).

The data cleansing function 42 performs cleansing such as removal of an abnormal value and interpolation of a missing value on the terminal use log and the weather information acquired in S100 (S101).

The hypertension detection model constructing function 44 constructs a hypertension detection model 44a based on training data in a predetermined period stored in the training data input database 40a (S102).

The life habit estimating function 43 estimates a life habit based on the terminal use log (S103). For example, the life habit estimating function 43 estimates a life habit based on the relationship illustrated in FIG. 5.

The hypertension detecting function 45 inputs the terminal use log, the life habit of the user whose hypertension is to be detected and the weather information of the user position to the hypertension detection model 44a, and the hypertension detection model 44a outputs blood pressure information which is a hypertension detection result (S104).

The result notifying function 46 notifies the user terminal of the hypertension detection result (S105). In this process of S102, the hypertension detection model 44a is constructed, but this process is not necessary. The hypertension detection model 44a may be constructed in advance and the process of S103 may be performed after the process of S101 has been performed.

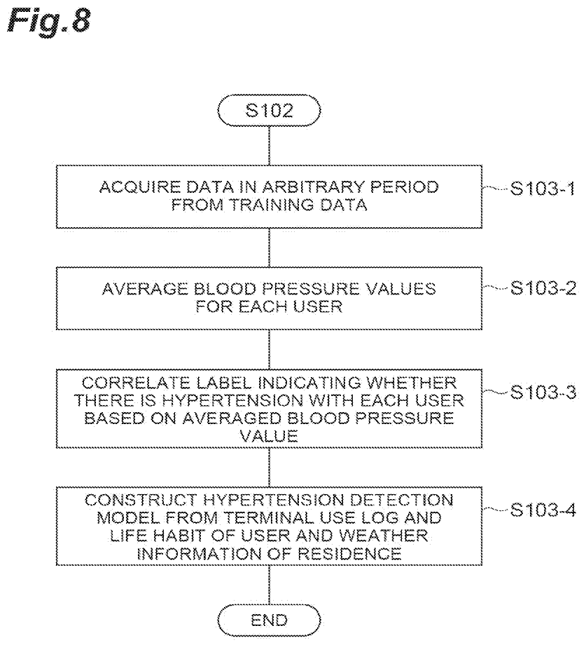

The process of constructing the hypertension detection model 44a in S102 will be described below in more detail. FIG. 8 is a flowchart illustrating a detailed process of the hypertension detection model constructing function 44. The hypertension detection model constructing function 44 acquires learning data (which includes learning use logs, learning life habit information, and learning blood information) such as user attributes, terminal use logs, life habit information, weather information, and blood information (blood pressure values herein) in an arbitrary period from training data stored in the training data input database 40a (S103-1).

Then, the hypertension detection model constructing function 44 averages a plurality of blood pressure values for each user in the acquired data (S103-2). The training data includes a plurality of pieces of time-series data (blood information) for the same user and excludes temporary abnormal values by calculating an average value thereof. A median value, a moving average value at an arbitrary time point, or the like may be used instead of the average value.

The hypertension detection model constructing function 44 correlates a label indicating whether there is hypertension (which may additionally include a degree of hypertension) with each user based on the averaged blood pressure value (S103-3). The hypertension detection model constructing function 44 constructs a hypertension detection model 44a for estimating the average blood pressure value or the hypertension label from the terminal use logs and the life habits of the users in the training data and weather information at the current location (S103-4). For example, the hypertension detection model constructing function 44 performs machine learning using the terminal use logs and the life habits of the users and the weather information at the current location as explanatory variables and using the blood pressure value or the hypertension label as an objective variable. In the present disclosure, a machine learning method which is used is not particularly limited. For example, a classical linear model may be used or a method such as SVM, XGBoost, or LightGBM or deep learning such as DNN may be used.

In this way, the hypertension detection model constructing function 44 can train the hypertension detection model 44a based on the terminal use logs of the user terminals.

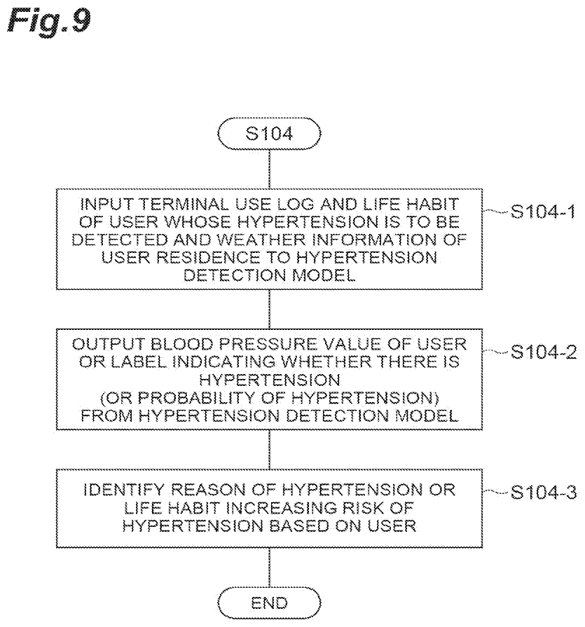

The process of S104 which is a hypertension detecting process using the hypertension detection model 44a will be described below. FIG. 9 is a flowchart illustrating the process. The hypertension detecting function 45 inputs the terminal use logs and the life habits of a user whose hypertension is to be detected and weather information at a user location to the hypertension detection model 44a (S104-1). Here, the life habits are information estimated in S103.

The hypertension detecting function 45 receives a blood pressure value of a corresponding user or a label indicating whether there is hypertension (or a probability or a blood pressure value of hypertension) from the hypertension detection model 44a (S104-2).

The hypertension detecting function 45 identifies a reason of hypertension or a life habit which can cause an increase of a risk of hypertension for the user (S104-3). The hypertension detection model constructing function 44 may identify the life habit which can cause an increase of a risk of hypertension based on magnitudes of coefficients associated with features (for example, weighted coefficients of an intermediate layer in machine learning) of the hypertension detection model 44a or may identify the life habit from an index for evaluating a degree of significance of features using a method of analyzing a prediction model of machine learning such as LIME or SHAP. For example, when the user has hypertension (or a high probability of hypertension), the hypertension detecting function 45 estimates what life habit has affected the output of the hypertension detection model constructing function 44, that is, the label indicating whether there is hypertension (or a probability or a blood pressure value of hypertension), such as whether an amount of motion of the user is small or whether stress of the user is high.

Operational advantages of the blood information estimating apparatus 100 according to the present disclosure will be described below. The blood information estimating apparatus 100 according to the present disclosure includes the data acquiring function 41 serving as a log acquiring unit configured to acquire a terminal use log of a user terminal such as a mobile terminal and the hypertension detecting function 45 serving as an estimation unit configured to estimate blood information on change of a blood status based on the terminal use log.

With this configuration, it is possible to estimate a blood status (a blood pressure, a blood-sugar level, a triglycerides level, or a cholesterol level) without imposing an examination burden on a user. Accordingly, it is possible to simply estimate a status such as masked hypertension.

In the aforementioned description, a blood status is estimated in consideration of user attribute information, weather information, and life habit information, but has only to be estimated using at least the terminal use log.

In the blood information estimating apparatus 100 according to the present disclosure, the blood information may include at least one of a blood pressure, a blood-sugar level, a triglycerides level, and a cholesterol level.

The blood information estimating apparatus 100 according to the present disclosure may estimate blood information at least one of the number of steps, a sleeping time, position information, screen-on/off information, acceleration information, illuminance, browsing URL information, a purchase history, and used application information.

In the blood information estimating apparatus 100 according to the present disclosure, the life habit estimating function 43 may estimate life habit information including at least one of a quantity of motion, a movement time, calorie consumption, a sleeping time, a sleep quality, regularity, and stress based on the terminal use log and estimate the blood information based on the life habit information.

With this configuration, it is possible to estimate blood information in consideration of the life habits and to more accurately estimate blood information.

The blood information estimating apparatus 100 according to the present disclosure may further include a hypertension detection model for outputting blood information in response to the terminal use log. The hypertension detecting function 45 estimates blood information using the hypertension detection model.

In the blood information estimating apparatus 100 according to the present disclosure, the hypertension detection model constructing function 44 may construct a hypertension detection model by performing learning based on an average value of blood information in a predetermined period as blood information at the time of learning.

Blood information, particularly, a blood pressure, a blood-sugar level, triglycerides information, and cholesterol information, changes. Accordingly, when a hypertension detection model based on only one time point, it is not possible to perform accurate estimation. For example, when learning is performed based on blood information in only an examination room, it is possible to acquire accurate estimation results. According to the present disclosure, it is possible to enhance estimation accuracy by performing learning based on an average value of blood information.

The hypertension detection model constructing function 44 according to the present disclosure may train the prediction model based on terminal use logs and blood information in predetermined age and/or sex, and the hypertension detecting function 45 may estimate the blood information using the hypertension detection model based on age and/or sex of a user.

With this configuration, it is possible to perform estimation with high accuracy using a model based on age and sex.

The hypertension detecting function 45 according to the present disclosure identifies a life habit serving as a basis of the estimated blood information. For example, when it is determined that there is hypertension, a life habit contributing to this determination is identified. The hypertension detecting function 45 can identify the life habit using an analysis technique (LIME or SHAP) used for the hypertension detection model 44a to perform analysis.

Accordingly, it is possible to prompt the user to improve the life habit serving as a reason why it is determined that there is hypertension.

The block diagram used for the description of the above embodiments shows blocks of functions. Those functional blocks (component parts) are implemented by any combination of at least one of hardware and software. Further, a means of implementing each functional block is not particularly limited. Specifically, each functional block may be implemented by one physically or logically combined device or may be implemented by two or more physically or logically separated devices that are directly or indirectly connected (e.g., by using wired or wireless connection etc.). The functional blocks may be implemented by combining software with the above-described one device or the above-described plurality of devices.

The functions include determining, deciding, judging, calculating, computing, processing, deriving, investigating, looking up/searching/inquiring, ascertaining, receiving, transmitting, outputting, accessing, resolving, selecting, choosing, establishing, comparing, assuming, expecting, considering, broadcasting, notifying, communicating, forwarding, configuring, reconfiguring, allocating/mapping, assigning and the like, though not limited thereto. For example, the functional block (component part) that implements the function of transmitting is referred to as a transmitting unit or a transmitter. In any case, a means of implementation is not particularly limited as described above.

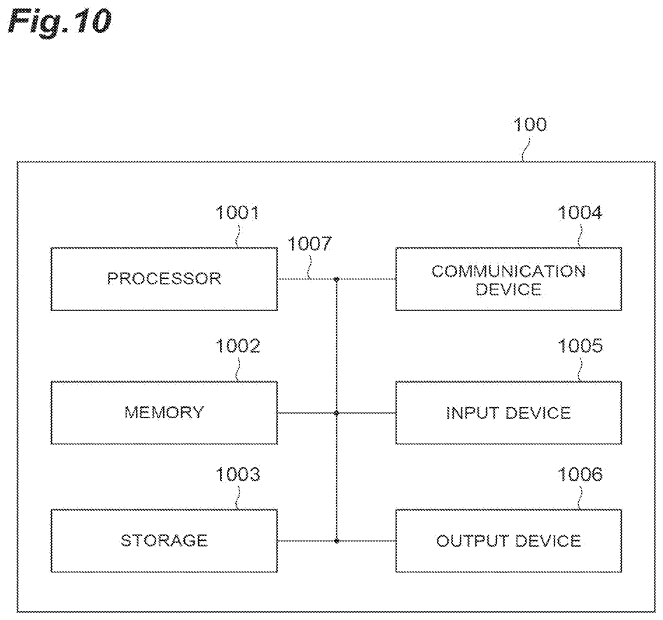

For example, the blood information estimating apparatus 100 according to one embodiment of the present disclosure may function as a computer that performs processing of a blood information estimating method in an interactive process according to the present disclosure. FIG. 10 is a view showing an example of the hardware configuration of the blood information estimating 100 according to one embodiment of the present disclosure. The blood information estimating 100 described above may be physically configured as a computer device that includes a processor 1001, a memory 1002, a storage 1003, a communication device 1004, an input device 1005, an output device 1006, a bus 1007 and the like.

In the following description, the term “device” may be replaced with a circuit, a device, a unit, or the like. The hardware configuration of the blood information estimating 100 may be configured to include one or a plurality of the devices shown in the drawings or may be configured without including some of those devices.

The functions of the blood information estimating 100 may be implemented by loading predetermined software (programs) on hardware such as the processor 1001 and the memory 1002, so that the processor 1001 performs computations to control communications by the communication device 1004 and control at least one of reading and writing of data in the memory 1002 and the storage 1003.

The processor 1001 may, for example, operate an operating system to control the entire computer. The processor 1001 may be configured to include a CPU (Central Processing Unit) including an interface with a peripheral device, a control device, an arithmetic device, a register and the like. For example, the terminal use log acquiring unit the hypertension detecting unit 40, and the like described above may be implemented by the processor 1001.

Further, the processor 1001 loads a program (program code), a software module and data from at least one of the storage 1003 and the communication device 1004 into the memory 1002 and performs various processing according to them. As the program, a program that causes a computer to execute at least some of the operations described in the above embodiments is used. For example, the hypertension detecting unit the like may be implemented by a control program that is stored in the memory 1002 and operates on the processor 1001, and the other functional blocks may be implemented in the same way. Although the above-described processing is executed by one processor 1001 in the above description, the processing may be executed simultaneously or sequentially by two or more processors 1001. The processor 1001 may be implemented in one or more chips. Note that the program may be transmitted from a network through a telecommunications line.

The memory 1002 is a computer-readable recording medium, and it may be composed of at least one of ROM (Read Only Memory), EPROM (ErasableProgrammable ROM), EEPROM (Electrically ErasableProgrammable ROM), RANI (Random Access Memory) and the like, for example. The memory 1002 may be also called a register, a cache, a main memory (main storage device) or the like. The memory 1002 can store a program (program code), a software module and the like that can be executed for implementing a blood information estimating method according to one embodiment of the present disclosure.

The storage 1003 is a computer-readable recording medium, and it may be composed of at least one of an optical disk such as a CD-ROM (Compact Disk ROM), a hard disk drive, a flexible disk, a magneto-optical disk (e.g., a compact disk, a digital versatile disk, and a Blu-ray (registered trademark) disk), a smart card, a flash memory (e.g., a card, a stick, and a key drive), a floppy (registered trademark) disk, a magnetic strip and the like, for example. The storage 1003 may be called an auxiliary storage device. The above-described storage medium may be a database, a server, or another appropriate medium including the memory 1002 and/or the storage 1003, for example.

The communication device 1004 is hardware (a transmitting and receiving device) for performing communication between computers via at least one of a wired network and a wireless network, and it may also be referred to as a network device, a network controller, a network card, a communication module, or the like. The communication device 1004 may include a high-frequency switch, a duplexer, a filter, a frequency synthesizer or the like in order to implement at least one of FDD (Frequency Division Duplex) and TDD (Time Division Duplex), for example. For example, the above-described terminal use log acquiring unit 20 or the like may be implemented by the communication device 1004.

The input device 1005 is an input device (e.g., a keyboard, a mouse, a microphone, a switch, a button, a sensor, etc.) that receives an input from the outside. The output device 1006 is an output device (e.g., a display, a speaker, an LED lamp, etc.) that makes output to the outside. Note that the input device 1005 and the output device 1006 may be integrated (e.g., a touch panel).

In addition, the devices such as the processor 1001 and the memory 1002 are connected by the bus 1007 for communicating information. The bus 1007 may be a single bus or may be composed of different buses between different devices.

Further, the blood information estimating apparatus 100 may include hardware such as a microprocessor, a DSP (Digital Signal Processor), an ASIC (Application Specific Integrated Circuit), a PLD (Programmable Logic Device), and an FPGA (Field Programmable Gate Array), and some or all of the functional blocks may be implemented by the above-described hardware components. For example, the processor 1001 may be implemented with at least one of these hardware components.

Notification of information may be made by another method, not limited to the aspects/embodiments described in the present disclosure. For example, notification of information may be made by physical layer signaling (e.g., DCI (Downlink Control Information), UCI (Uplink Control Information)), upper layer signaling (e.g., RRC (Radio Resource Control) signaling, MAC (Medium Access Control) signaling, annunciation information (MIB (Master Information Block), SIB (System Information Block))), another signal, or a combination of them. Further, RRC signaling may be called an RRC message, and it may be an RRC Connection Setup message, an RRC Connection Reconfiguration message or the like, for example.

The procedure, the sequence, the flowchart and the like in each of the aspects/embodiments described in the present disclosure may be in a different order unless inconsistency arises. For example, for the method described in the present disclosure, elements of various steps are described in an exemplified order, and it is not limited to the specific order described above.

Input/output information or the like may be stored in a specific location (e.g., memory) or managed in a management table. Further, input/output information or the like can be overwritten or updated, or additional data can be written. Output information or the like may be deleted. Input information or the like may be transmitted to another device.

The determination may be made by a value represented by one bit (0 or 1), by a truth-value (Boolean: true or false), or by numerical comparison (e.g., comparison with a specified value).

Each of the aspects/embodiments described in the present disclosure may be used alone, may be used in combination, or may be used by being switched according to the execution. Further, a notification of specified information (e.g., a notification of “being X”) is not limited to be made explicitly, and it may be made implicitly (e.g., a notification of the specified information is not made).

Although the present disclosure is described in detail above, it is apparent to those skilled in the art that the present disclosure is not restricted to the embodiments described in this disclosure. The present disclosure can be implemented as a modified and changed form without deviating from the spirit and scope of the present disclosure defined by the appended claims. Accordingly, the description of the present disclosure is given merely by way of illustration and does not have any restrictive meaning to the present disclosure.

Software may be called any of software, firmware, middleware, microcode, hardware description language or another name, and it should be interpreted widely so as to mean an instruction, an instruction set, a code, a code segment, a program code, a program, a sub-program, a software module, an application, a software application, a software package, a routine, a sub-routine, an object, an executable file, a thread of execution, a procedure, a function and the like.

Further, software, instructions and the like may be transmitted and received via a transmission medium. For example, when software is transmitted from a website, a server or another remote source using at least one of wired technology (a coaxial cable, an optical fiber cable, a twisted pair and a digital subscriber line (DSL) etc.) and wireless technology (infrared rays, microwave etc.), at least one of those wired technology and wireless technology are included in the definition of the transmission medium.

The information, signals and the like described in the present disclosure may be represented by any of various different technologies. For example, data, an instruction, a command, information, a signal, a bit, a symbol, a chip and the like that can be referred to in the above description may be represented by a voltage, a current, an electromagnetic wave, a magnetic field or a magnetic particle, an optical field or a photon, or an arbitrary combination of them.

Note that the term described in the present disclosure and the term needed to understand the present disclosure may be replaced by a term having the same or similar meaning. For example, at least one of a channel and a symbol may be a signal (signaling). Further, a signal may be a message. Furthermore, a component carrier (CC) may be called a cell, a frequency carrier, or the like.

Further, the information, parameters and the like described in the present disclosure may be expressed using absolute values, relative values from a predetermined value, or other corresponding information.

Note that the term “determining” and “determining” used in the present disclosure includes a variety of operations. For example, “determining” and “determining” can include regarding the act of judging, calculating, computing, processing, deriving, investigating, looking up/searching/inquiring (e.g., looking up in a table, a database or another data structure), ascertaining or the like as being “determined” and “determined”. Further, “determining” and “determining” can include regarding the act of receiving (e.g., receiving information), transmitting (e.g., transmitting information), inputting, outputting, accessing (e.g., accessing data in a memory) or the like as being “determined” and “determined”. Further, “determining” and “determining” can include regarding the act of resolving, selecting, choosing, establishing, comparing or the like as being “determined” and “determined”. In other words, “determining” and “determining” can include regarding a certain operation as being “determined” and “determined”. Further, “determining (determining)” may be replaced with “assuming”, “expecting”, “considering” and the like.

The term “connected”, “coupled” or every transformation of this term means every direct or indirect connection or coupling between two or more elements, and it includes the case where there are one or more intermediate elements between two elements that are “connected” or “coupled” to each other. The coupling or connection between elements may be physical, logical, or a combination of them. For example, “connect” may be replaced with “access”. When used in the present disclosure, it is considered that two elements are “connected” or “coupled” to each other by using at least one of one or more electric wires, cables, and printed electric connections and, as several non-definitive and non-comprehensive examples, by using electromagnetic energy such as electromagnetic energy having a wavelength of a radio frequency region, a microwave region and an optical (both visible and invisible) region.

The description “on the basis of” used in the present disclosure does not mean “only on the basis of” unless otherwise noted. In other words, the description “on the basis of” means both of “only on the basis of” and “at least on the basis of”.

Furthermore, “means” in the configuration of each device described above may be replaced by “unit”, “circuit”, “device” or the like.

As long as “include”, “including” and transformation of them are used in the present disclosure, those terms are intended to be comprehensive like the term “comprising”. Further, the term “or” used in the present disclosure is intended not to be exclusive OR.

In the present disclosure, when articles, such as “a”, “an”, and “the” in English, for example, are added by translation, the present disclosure may include that nouns following such articles are plural.

In the present disclosure, the term “A and B are different” may mean that “A and B are different from each other”. Note that this term may mean that “A and B are different from C”. The terms such as “separated” and “coupled” may be also interpreted in the same manner.

### REFERENCE SIGNS LIST

100 Blood information estimating apparatus
20 Terminal use log acquiring unit
30 Weather information acquiring unit
40 Hypertension detecting unit
21 User authenticating function
22 User attribute information acquiring function
23 Application use information acquiring function
24 Purchase history information acquiring function
25 Position information acquiring function
26 Terminal operation information acquiring function
27 Data transmitting function
31 User authenticating function
32 User position information acquiring function
33 Weather information acquiring function
34 Data transmitting function
30a Weather information database
41 Data acquiring function
42 Data cleansing function
43 Life habit estimating function
44 Hypertension detection model constructing function
45 Hypertension detecting function
46 Notification function
40a Training data input database
44a Hypertension detection model

1. A blood information estimating apparatus comprising:
a log acquiring unit configured to acquire a use log of a user terminal; and
a blood information estimating unit configured to estimate blood information on change of a blood status based on the use log.

2. The blood information estimating apparatus according to claim 1, wherein the blood information includes at least one of a blood pressure, a blood-sugar level, a triglycerides level, and a cholesterol level.

3. The blood information estimating apparatus according to claim 1, wherein the blood information estimating unit estimates the blood information in consideration of at least one of user attribute information, weather information, or life habit information.

4. The blood information estimating apparatus according to claim 1, wherein the blood information estimating unit estimates life habit information including at least one of a quantity of motion, commuting means, time, and pattern, stress, a degree of fatigue, a degree of happiness, regularity of life and sleep, an eating-out frequency, a salt intake, and a calorie intake of a user based on the use log and estimates the blood information based on the life habit information.

5. The blood information estimating apparatus according to claim 1, wherein the use log includes at least one of the number of steps, a sleeping time, a weight, a body temperature, a pulse rate, a camera image, purchase store information, a purchase price, a purchased commodity, GPS information, base station service information, screen-on/off information, acceleration information, gyro information, illuminance, browsing URL information, and used application information.

6. The blood information estimating apparatus according to claim 1, further comprising a blood information prediction model that outputs the blood information based on the use log,
wherein the blood information estimating unit estimates the blood information using the prediction model.

7. The blood information estimating apparatus according to claim 6, further comprising a learning unit configured to train the prediction model based on learning use logs and learning blood information stored as training data,
wherein the learning blood information at the time of training in the learning unit is information based on an average value in a predetermined period.

8. The blood information estimating apparatus according to claim 6, wherein the blood information estimating unit identifies a life habit that affects the blood information estimated by the prediction model.

9. The blood information estimating apparatus according to claim 6, wherein the prediction model is trained based on the use log and the blood information in predetermined age and/or sex, and
wherein the blood information estimating unit estimates the blood information using the prediction model based on age and/or sex of a user.

10. The blood information estimating apparatus according to claim 12, wherein the blood information estimating unit estimates the blood information in consideration of at least one of user attribute information, weather information, or life habit information.

11. The blood information estimating apparatus according to claim 2, wherein the blood information estimating unit estimates life habit information including at least one of a quantity of motion, commuting means, time, and pattern, stress, a degree of fatigue, a degree of happiness, regularity of life and sleep, an eating-out frequency, a salt intake, and a calorie intake of a user based on the use log and estimates the blood information based on the life habit information.

12. The blood information estimating apparatus according to claim 3, wherein the blood information estimating unit estimates life habit information including at least one of a quantity of motion, commuting means, time, and pattern, stress, a degree of fatigue, a degree of happiness, regularity of life and sleep, an eating-out frequency, a salt intake, and a calorie intake of a user based on the use log and estimates the blood information based on the life habit information.

13. The blood information estimating apparatus according to claim 2, wherein the use log includes at least one of the number of steps, a sleeping time, a weight, a body temperature, a pulse rate, a camera image, purchase store information, a purchase price, a purchased commodity, GPS information, base station service information, screen-on/off information, acceleration information, gyro information, illuminance, browsing URL information, and used application information.

14. The blood information estimating apparatus according to claim 3 wherein the use log includes at least one of the number of steps, a sleeping time, a weight, a body temperature, a pulse rate, a camera image, purchase store information, a purchase price, a purchased commodity, GPS information, base station service information, screen-on/off information, acceleration information, gyro information, illuminance, browsing URL information, and used application information.

15. The blood information estimating apparatus according to claim 7, wherein the blood information estimating unit identifies a life habit that affects the blood information estimated by the prediction model.

16. The blood information estimating apparatus according to claim 7, wherein the prediction model is trained based on the use log and the blood information in predetermined age and/or sex, and
wherein the blood information estimating unit estimates the blood information using the prediction model based on age and/or sex of a user.

17. The blood information estimating apparatus according to claim 6, wherein the prediction model is trained based on the use log and the blood information in predetermined age and/or sex, and
wherein the blood information estimating unit estimates the blood information using the prediction model based on age and/or sex of a user.
